(19) United States
(12) Patent Application Publication
(10) Pub. No.: US 2026/0265695 A1
(43) Pub. Date: Sep. 10, 2026

(54) GENERATION OF INDUCED NEUTROPHILS WITH INCREASED ACTIVATION AND TRANSMIGRATION CAPABILITIES

(71) Applicant: Wisconsin Alumni Research Foundation, Madison, WI (US)

(72) Inventors: Anna Huttenlocher, Madison, WI (US); David Bennin, Mount Horeb, WI (US); Frances Smith, Madison, WI (US)

(21) Appl. No.: 19/380,658

(22) Filed: Nov. 5, 2025

Related U.S. Application Data
(60) Provisional application No. 63/717,076, filed on Nov. 6, 2024.

(51) Int. Cl. C12N 5/0787; A61K 35/15; A61P 31/04; C07K 14/475; C12N 9/22; C12N 15/10

(52) U.S. Cl. CPC C12N 5/0642; A61K 35/15; A61P 31/04; C07K 14/475; C12N 9/226; C12N 15/102; C12N 2500/90; C12N 2500/98; C12N 2501/115; C12N 2501/165; C12N 2501/22; C12N 2501/385; C12N 2506/45; C12N 2510/00

(57) ABSTRACT

This disclosure provides methods for producing neutrophils under serum-free and feeder-free conditions from myeloid derived growth factor (MYDGF)-depleted pluripotent stem cells. The disclosure also provides populations of neutrophils produced thereby having enhanced anti-microbial function, wherein the neutrophils have inhibited expression of myeloid derived growth factor (MYDGF). The produced neutrophils have enhanced anti-microbial function and increased ability to cross blood vessel compared to neutrophils derived from pluripotent stem cells without inhibited MYDGF expression. Also disclosed are pharmaceutical compositions comprising neutrophils produced by these methods or neutrophils with inhibited expression of MYDGF, and a pharmaceutically acceptable carrier, as well as methods for using these pharmaceutical compositions to treat a microbial infection or an inflammatory disease in a subject.

### CROSS-REFERENCE TO RELATED APPLICATIONS

This application claims priority to U.S. provisional application No. 63/717,076, filed Nov. 6, 2024, the disclosure of which is expressly incorporated by reference herein.

### STATEMENT REGARDING FEDERALLY SPONSORED RESEARCH

This invention was made with government support under GM118027 awarded by the National Institutes of Health. The government has certain rights in the invention.

### REFERENCE TO AN ELECTRONIC SEQUENCE LISTING

This application contains a Sequence Listing that has been submitted electronically and is hereby incorporated by reference in its entirety. The Sequence Listing was created on Oct. 7, 2025, is named “24-1058-US_SequenceListing.xml,” and is 12,825 bytes in size.

### FIELD OF THE DISCLOSURE

This disclosure provides a population of neutrophils with enhanced anti-microbial function, wherein the neutrophils have inhibited expression of myeloid derived growth factor (MYDGF). The disclosure also provides methods for producing neutrophils under serum-free and feeder-free conditions from myeloid derived growth factor (MYDGF)-inhibited pluripotent stem cells. The produced neutrophils have enhanced anti-microbial function compared to neutrophils derived from pluripotent stem cells without inhibited MYDGF expression. Also disclosed are pharmaceutical compositions comprising neutrophils produced by these methods or neutrophils with inhibited expression of MYDGF, and a pharmaceutically acceptable carrier, as well as methods for using these pharmaceutical compositions to treat a microbial infection in a subject.

### BACKGROUND OF THE DISCLOSURE

Neutrophils are the initial responders to infection and injury in mammals and are critical for host survival. Immunocompromised patients are prone to recurrent infections that can require interventions such as granulocyte transfusions. However, these transfusions have minimal efficacy as donor material is limited and highly heterogeneous. Induced pluripotent stem cells (iPSCs) are a promising option for generating a defined therapeutic cellular agent that can improve patient outcomes. However, iPSC-derived neutrophils have been shown to have reduced antimicrobial function in comparison to primary human neutrophils.

Thus, there remains a need for efficient and cost-effective protocols for generating iPSC-derived neutrophils with enhanced anti-microbial function.

### SUMMARY

The disclosure provides a population of neutrophils derived from human pluripotent stem cells having inhibited expression of myeloid derived growth factor (MYDGF), wherein the neutrophils have enhanced anti-microbial function compared to neutrophils derived from pluripotent stem cells not having inhibited expression of MYDGF.

Also provided herein is a method of producing neutrophils having inhibited expression of MYDGF from pluripotent stem cells (PSCs) comprising inhibiting expression of MYDGF in the PSCs and differentiating the PSCs into the neutrophils.

In some embodiments, differentiating the PSCs into neutrophils comprises:
(a) transiently introducing exogenous ETV2 in the human pluripotent stem cells having inhibited expression of MYDGF and culturing the ETV2-pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors; and (c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils.

In some embodiments, the serum-free and xeno-free culture medium in (b) further comprises UM171. In some embodiments, the retinoic acid receptor agonist is AM580. In some embodiments, the method comprises one or more of the following: step (a) comprises culturing the ETV2-pluripotent stem cells for about 1 to 2 days; step (b) comprises culturing the ETV2-HECs for about 7 to 12 days; and steps (c) and (d) comprises culturing the myeloid progenitor cells for about 6 to 8 days. In some embodiments, the pluripotent stem cells are induced pluripotent stem cells.

In some embodiments, the described method further comprises step (d) selecting mature neutrophils from the population of neutrophils. The mature neutrophils express CD15 and/or CD16. In some embodiments, selecting comprises a cell separation, cell sorting, or enrichment method.

In some embodiments, the expression of MYDGF is inhibited by deletion of exon 4 of the MYDGF gene. In some embodiments, the expression of MYDGF is inhibited using gene mutation, RNA-mediated inhibition, RNA editing, or DNA gene editing or base editing. Examples of gene editing methods include sequence modification using a nuclease selected from a meganuclease, ZFNs, TALENs, or Cas enzyme. In some embodiments, the nuclease is a Cas9 enzyme.

Also provided herein is a pharmaceutical composition comprising a therapeutically effective amount of the population of neutrophils having enhanced anti-microbial function as described above and a pharmaceutically acceptable carrier.

In another aspect, the disclosure provides a method of treating a microbial injection in a subject comprising administering the pharmaceutical composition to the subject. In some embodiments, the microbial infection is a bacterial or fungal infection. In some embodiments, the microbial infection is a systemic infection. In some embodiments, the pharmaceutical composition is administered intravenously to the subject. In some embodiments, the subject is a patient that has neutrophil deficiency, autoimmune disease, or neutrophil depletion due to radiation or chemotherapy treatment.

In some embodiments, the pharmaceutical composition is administered intravenously to the subject. In some embodiments, the subject is a patient that has neutrophil deficiency, autoimmune disease, or neutrophil depletion due to radiation or chemotherapy treatment.

These and other features, objects, and advantages of the present invention will become better understood from the description that follows. In the description, reference was made to the accompanying drawings, which form a part hereof and in which there was shown by way of illustration, not limitation, embodiments of the invention. The description of preferred embodiments was not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration was given to the following detailed description thereof. Such detailed description refers to the following drawings.

FIG. 1A is a diagram of a differentiation timeline of human iPSCs into iPSC-derived neutrophils (termed herein “iNeutrophils”).

FIG. 1B shows an increase in MYDGF expression in zebrafish following tailfin transection, most notably in neutrophils (figure adapted from Houseright et al., 2021, J Cell Biol. 220 (8): e202103054).

FIG. 1C shows Western blot analysis confirming successful deletion of human MYDGF in iPSC lines. Mouse tubulin is included as a protein control. rbαhMYDGF: rabbit anti-human MYDGF antibody. msαb Tubulin: mouse anti-beta tubulin antibody.

FIG. 2A shows cytospin images of wild type (WT) and MYDGF knockout (KO) iNeutrophils, having nuclei stained with Differential Quick (Diffquik) III Stain Kit (obtained from Electron Microscopy Sciences) (scale bar=100 μm).

FIG. 2B shows quantitative data of number of cell clusters and the clusters' area in the cytospin images. (n=8).

FIG. 2C shows flow cytometry-based verification of CD11b neutrophil marker on wild-type (WT) and MYDGF KO iNeutrophils. (n=3).

FIG. 2D shows expression of CD15 and CD16 neutrophil maturity markers by WT and MYDGF KO iNeutrophils. Cells initially gated using live/dead staining and live cells were percent quantified for CD11b+ population size. (n=3).

FIG. 2E is a Uniform Manifold Approximation and Projection (UMAP) plot showing expression of clusters of marker genes as indicated.

FIG. 2F shows percentage of cells expressing the marker genes as indicated.

FIG. 3A shows Western blot analysis confirming successful knock in of human MYDGF in the MYDGF KO iPSC lines. Mouse actin is included as a protein control. rbαhMYDGF: rabbit anti-human MYDGF antibody. msαActin: mouse anti-alpha actin antibody.

FIG. 3B shows quantification of fluorescence-based (DHR123) reactive oxygen species (ROS) of WT, MYDGF KO, and MYDGF rescue in KO iNeutrophils at baseline and in response to 100 μg/mL of Zymosan. (n=3)

FIG. 3C shows expression of pro-inflammatory cytokines IL-8 and TNFα measured by ELISA after stimulation with 100 ng/ml lipopolysaccharide (LPS) and 100 μg/ml Zymosan (n=6).

FIG. 4A shows images of WT and MYDGF KO iNeutrophils co-cultured with Aspergillus fumigatus at 0 hour and 12-hour time points; scale bar=50 μm. The circles indicate the fluorescent fungal growth.

FIG. 4B is quantification of fungal area at 12 hours after incubation with fungi in FIG. 4A.

FIG. 5A shows transendothelial lumen model of neutrophil migration wherein WT and MYDGF KO iNeutrophils migrate across an endothelial barrier in response to Pseudomonas aeruginosa.

FIG. 5B shows quantification of number of cells migrated out of the endothelial lumen in FIG. 5A.

FIG. 5C shows confined mechanical model of neutrophil migration through a 3 μm gap towards a chemoattractant, N-formyl-Met-Leu-Phe (fMLP).

FIG. 5D shows quantification of the speed in which the WT, MYDGF KO, and MYDGF rescue iNeutrophils migrate in FIG. 5C (n=6-7).

FIG. 5E shows Gene Ontology (GO) terms enriched in genes highly expressed in MYDGF-KO Neutrophils compared to WT by subpopulation from single-cell transcriptomes.

FIG. 6A shows fluorescence of Fluo4 calcium dye in a single calcium transient event during confined migration in FIG. 5C in a WT and a MYDGF KO iNeutrophil.

FIG. 6B shows quantification of the calcium fluorescence intensity in FIG. 6A.

FIG. 6C shows fluorescence of Fluo4 calcium dye in a population of WT and MYDGF KO iNeutrophils during confined migration.

FIG. 6D shows quantification of the fluorescence intensity in MYDGF KO and rescue iNeutrophils (n=3 each) in the population in FIG. 6C.

FIG. 6E shows fluorescence of Fluo4 calcium dye in a population of MYDGF KO iNeutrophils with and without BAPTA-AM, calcium chelator (n=3) treatment during confined migration.

FIG. 6F shows quantification of the calcium fluorescence intensity during confined migration in MYDGF KO iNeutrophils with or without BAPTA treatment (n=3) in the population in FIG. 6E.

FIG. 7A shows flow cytometry-based analysis of pattern recognition receptors (PRRs) Dectin-1, TLR2, and TLR4 on WT and MYDGF KO iNeutrophils (n=3).

FIG. 7B shows flow cytometry-based phagocytosis assay using pHrodo E. coli beads; gated on CD11b+ population (n=4).

FIG. 7C shows oxygen consumption rate (OCR) in WT and MYDGF KO iNeutrophils response to stimulation by PMA. (n=3).

FIG. 7D shows differentially expressed metabolites in WT and MYDGF KO iNeutrophils in response to zymosan. The heatmap shows colors associated with upregulation (above 0) and downregulation (below 0) compared to controls (without zymosan stimulation). (n=1)

### DETAILED DESCRIPTION OF THE DISCLOSURE

Provided herein are methods for producing neutrophils under serum-free and feeder-free conditions from myeloid derived growth factor (MYDGF)-depleted pluripotent stem cells. The disclosure also provides populations of neutrophils produced thereby having enhanced ability to transmigrate across endothelium and anti-microbial function, wherein the neutrophils have inhibited expression of myeloid derived growth factor (MYDGF). The produced neutrophils have enhanced anti-microbial function compared to neutrophils derived from pluripotent stem cells without inhibited MYDGF expression. Also disclosed are pharmaceutical compositions comprising neutrophils produced by these methods or neutrophils with inhibited expression of MYDGF, and a pharmaceutically acceptable carrier, as well as methods for using these pharmaceutical compositions to treat a microbial infection or an inflammatory disease in a subject.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation

### 1. Definitions

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” can be replaced with either of the other two terms, while retaining their ordinary meanings

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Subject” or “patient” as used herein are used interchangeably and refer to a warm-blooded animal such as a mammal, preferably a human, which is afflicted with, or has the potential to be afflicted with infection as described herein.

“Express” or “expression” as used herein refers to transcription and translation of a nucleic acid coding sequence resulting in production of the encoded polypeptide.

“Pluripotent stem cells” refers to cells having the capacity to differentiate into cells of all three germ layers. “Embryonic stem cells” or “ESCs” mean a pluripotent cell or population of pluripotent cells derived from an inner cell mass of a blastocyst. See Thomson et al., (1998) Science 282:1145-1147.

“Induced pluripotent stem cells” or “iPSCs” refer to a pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as embryonic stem cells (ESCs). See, e.g., Yu et al., (2007) Science 318:1917-1920.

“Induced neutrophils” or iNeutrophils refers to neutrophils that are differentiated from pluripotent stem cells.

“Hemogenic endothelial cells (HECs)” refers to a subset of endothelial cells that can differentiate into hematopoietic cells.

“Myeloid progenitors” are cells capable of differentiating into cell types of the myeloid lineages.

“Chemically defined culture,” “fully defined, growth factor free culture conditions,” and “fully defined conditions” indicate that the identity and quantity of each medium ingredient is known and the identity and quantity of supportive surface is known.

“Xeno-free culture medium” refers to medium that does not contain any components derived from animal sources, such as, for example, serum.

“Feeder-free” conditions refers to a pluripotent stem cell culture without feeder cells. Feeder cells are a layer of cells that support the growth of other cells such as pluripotent stem cells, by providing nutrients, growth factors, and extracellular secretions.

“Serum-free” refers to cell culture materials that are free of serum obtained from animal (e.g., fetal bovine) or human blood.

“DNA gene editing” refers to a method of making specific changes to the DNA or RNA of a cell or organism. These changes include but are not limited to insertion, deletion, and replacement of sequences in DNA or RNA. “Base editing” refers to a genome editing approach that uses components from CRISPR systems together with other enzymes to directly install point mutations into cellular DNA or RNA without making double-stranded DNA breaks. The gene editing method comprises sequence modification using a nuclease including, but not limited to, a meganuclease, zinc-finger nuclease (ZFN), transcription activator-like effector nuclease (TALEN), or Cas enzyme.

As used herein, the term “neutrophils derived from human pluripotent stem cells” refers to neutrophils that are differentiated or produced from human pluripotent stem cells.

Unless otherwise defined, all technical terms used herein have the same meaning as commonly understood by a person of ordinary skill in the art to which this disclosure belongs.

### 2. MYDGF Knock Out (KO) Induced Neutrophils (iNeutrophils)

Provided herein is a population of neutrophils derived from pluripotent stem cells having inhibited expression of myeloid derived growth factor (MYDGF). These induced neutrophils have enhanced anti-microbial function compared to neutrophils derived from pluripotent stem cells not having inhibited expression of MYDGF.

Neutrophils are a type of white blood cell that is an important part of the immune system and helps the body fight infection. Induced neutrophils derived from pluripotent stem cells are of great interest because, as opposed to limited sources of primary neutrophils, these neutrophils can be produced robustly from hPSCs in vitro and administered to patients as therapeutic treatment against diseases. However, the therapeutic use of induced neutrophils has been hindered by their limited functions compared to primary neutrophils. Recent findings have shown that MYDGF is induced by tissue damage and regulates neutrophil motility to damage by not infected tissues in zebrafish larvae (U.S. Pat. No. 11,980,654B2 and Houseright et al., 2021, J Cell Biol. 220 (8): e202103054). In contrast with these findings, this disclosure shows that MYDGF KO induced neutrophils (derived from MYDGF KO PSCs) have enhanced transmigration ability across endothelium and anti-microbial activity as set forth herein. Example 1 demonstrates that MYDGF KO induced neutrophils exhibited superior properties towards fighting infection compared to wild-type induced neutrophils (derived from PSCs without MYDGF gene knockout, KO). Features of MYDGF KO induced neutrophils include being more immature, more pro-inflammatory, more efficient in transmigrating across endothelial barrier to the site of infection/pathogen presence, and better at inhibiting fungal growth compared to wild-type induced neutrophils. Example 2 shows that an increase in expression of pattern recognition receptors that detect pathogenic ligands and a shift toward glucose oxidative metabolism seemed to play a role in enhanced production of reactive oxygen species (ROS) and cytokines, and improvement in migration observed in MYDGF KO induced neutrophils.

As used herein, the term “anti-microbial function” refers to one or more biological functions that promote killing of microbes. Anti-microbial function in a neutrophil can include one or more enhanced abilities in transmigration across the endothelial barrier, secretion of pro-inflammatory cytokines, secretion of ROS, activation of processes in response to infection, or inhibition of microbial growth.

### 3. Method for Generating MYDGF KO iNeutrophils

Provided herein are methods for producing neutrophils having inhibited expression of MYDGF from pluripotent stem cells comprising inhibiting expression of MYDGF in the PSCs and differentiating the PSCs into neutrophils.

Methods for differentiating these PSCs into neutrophils used in this disclosure is described in U.S. Application Publication No. US20200385676A1, the contents of which are incorporated by reference in its entirety. However, other methods of differentiating PSCs into neutrophils can also be employed in combination with inhibiting expression of MYDGF in the PSCs. These methods include, but are not limited to, those methods disclosed in U.S. Application Publication Nos. US20230338420A1, US20230323301A1, US20240156963A1, and U.S. Pat. No. 11,980,654B2, the disclosures of each of which are expressly incorporated by reference herein in their entirety.

Briefly, the method described in U.S. Application Publication No. US20200385676A1 and in this disclosure uses direct programming with transient expression of transcription factor variant ETV2 (see Oh et al., 2015, “The ETS Factor, ETV2: a Master Regulator for Vascular Endothelial Cell Development,” Molecules and Cells. 38:1029-36), e.g., by addition of modified mRNA (mmRNA) of ETV2 into the human iPSCs (hiPSCs). Initially, hiPSCs were directly programmed into hemogenic endothelial cells (HECs) using ETV2 mmRNA which transiently produced ETV2 within the cells in serum-free culture medium comprising vascular endothelial growth factor-165 (VEGF-165) and fibroblast growth factor 2 (FGF2). Next, the hemogenic endothelial cells were then differentiated into myeloid progenitors in the presence of granulocyte-macrophage colony-stimulating factor (GM-CSF), FGF2, and optionally UM171 (the presence of UM171 in combination with GM-CSF and FGF2 increases the number of neutrophils produced by the methods). Myeloid progenitors which are non-adherent could be continuously collected from cultures every 8-10 days for up to 30 days of post ETV2 transfection. Finally, these myeloid progenitors were subsequently differentiated into neutrophils.

Provided herein are methods for producing neutrophils having inhibited expression of MYDGF, such methods comprising:
(a) transiently introducing exogenous ETV2 in human pluripotent stem cells having inhibited expression of MYDGF and culturing the ETV2-pluripotent stem cells in serum-free culture medium comprising Vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils.

In some embodiments, the method further comprises step (d) selecting mature neutrophils from the population of neutrophils.

The pluripotent stem cells and their derived neutrophils described herein have inhibited expression of MYDGF. The term “having inhibited expression of MYDGF,” indicates that expression of the MYDGF gene (see, MYDGF myeloid derived growth factor {Homo sapiens (human)}, Gene ID 56005; Accession No: NM_019107.4; SEQ ID NO: 7) is repressed or the protein encoded therein is not expressed in a functional protein form. In particular embodiments, expression of MYDGF is knocked out so that that there is no detectable expression of MYDGF in the PSCs and their derived neutrophils. This inhibition or knockout can be obtained by gene mutation, RNA-mediated inhibition, RNA editing, DNA gene editing or base editing.

Examples of gene editing components include RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-MLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3 (3): e155, 2022, each of which is incorporated by reference for all that they teach about the components and uses of this technology to the extent that it does not conflict with the present disclosure. In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme or derivatives thereof having an equivalent base editing function.

In some embodiments transient expression of ETV2 is achieved by introduction of modified mRNAs (mmRNAs) encoding ETV2 into hPSCs, e.g., by transfection or electroporation. mmRNAs and their synthesis is described in detail in, e.g., U.S. Patent Application Publication No 20120046346. Typically, mmRNAs comprise (i) a 5′ synthetic cap for enhanced translation; (ii) modified nucleotides that confer RNAse resistance and an attenuated cellular interferon response, which would otherwise greatly reduce translational efficiency; and (iii) a 3′ poly-A tail. Typically, ETV2 mmRNAs are synthesized in vitro from a DNA template comprising an SP6 or T7 RNA polymerase promoter-operably linked to an open reading frame encoding an ETV2. The mmRNA synthesis reaction is carried in the presence of a mixture of modified and unmodified nucleotides. In some embodiments modified nucleotides included in the in vitro synthesis of mmRNAs are pseudo-uridine and 5-methyl-cytosine. A key step in cellular mRNA processing is the addition of a 5′ cap structure, which is a 5′-5′ triphosphate linkage between the 5′ end of the RNA and a guanosine nucleotide. The cap is methylated enzymatically at the N-7 position of the guanosine to form mature mCAP. When preparing ETV2 mmRNAs, a 5′ cap is typically added prior to transfection of hPSCs in order to stabilize IF mmRNA and significantly enhance translation. In some embodiments a 4:1 mixture of a cap analog to GTP is used in transcription reactions to obtained 5′-capped mmRNAs. In preferred embodiments, the Anti Reverse Cap Analog (ARCA), 3′-O-Me-m7G(5′)ppp(5′)G is used to generate ETV2 mmRNAs that can be efficiently translated in hPSCs. Systems for in vitro synthesis are commercially available, as exemplified by the mRNAExpress™ mRNA Synthesis Kit (System Biosciences, Mountain View, Calif.). ETV2 mmRNAs can be introduced into hPSCs by any of a number of established methods for transfection of mammalian cells, e.g., electroporation, nucleoporation, or lipofection.

ETV2 can be transiently introduced into the PSCs by methods known in the art. Methods of transiently expressing ETV2 in PSCs include, but are not limited to, for example, introducing transiently exogenous nucleic acids encoding the protein of interest (e.g., by plasmid expression vector transfection, or modified mRNA transfection) or protein transduction, among others. In one embodiment, mmRNA of ETV-2 (e.g., Accession No: NM_014209.2; SEQ ID NO: 6) is introduced into PSCs by suitable methods as described in U.S. Pat. No. 9,382,531 and U.S. Application Publication No. US20200385676A1, the contents of each of which are incorporated by reference in its entirety. Methods of introducing mmRNA into PSCs are known in the art, and include, but are not limited to, transfection or electroporation. The methods of introducing mmRNA or DNA to transiently express ETV-2 protein is within the skill of one having ordinary skill in the art.

The term “introducing ETV2” encompasses transient expression of ETV2 (SEQ ID NO: 8), a protein comprising the ETV2 DNA binding domains (DBDs, SEQ ID NO: 9) and a heterologous transactivation domain (TDs) or a homolog thereof that have the same functionality of ETV2. The skilled artisan recognizes that in many cases it is possible to substitute a native transactivation domain with an unrelated transactivation domain (e.g., VP16, GAL4, or LEX TDs) well known in the art and often used to generate functional heterologous transcription factors having a desired DBD, e.g., the ETV2 DBD (SEQ ID NO: 9), and a heterologous TD, e.g. GATA1 (DBD)-VP16, as exemplified in Blobel et al. (1995), Mol Cell Biol, 15 (2): 626-633. The amino acid sequence of the VP16 transactivation domain (SEQ ID NO: 10). The present methods contemplate the ability to use any function homologs of ETV2 in the practice of the methods. A functional homolog may comprise the amino acid sequence of an artificial transcription factor that has no significant sequence identity to ETV2 (SEQ ID NO: 8), but is able to bind and transactivate a cognate promoter sequence. For example, the artificial DBD may be generated by designing zinc finger-containing proteins having binding specificity for a designed target sequence (e.g., an ETV2 motif). The zinc-finger DBD is then fused to a transactivator protein, e.g., VP16 to generate a fusion protein that is an artificial TF. See, e.g., Wilson et al (2013), Mol Ther Nucleic Acids, (published online): 2, e87; doi: 10.1038; and Klug (2010), Q Rev Biophys.; February; 43 (1): 1-21. doi: 10.1017/50033583510000089.

The disclosed methods also contemplate using ETV proteins or a sequence with at least 75% identity to ETV2 (SEQ ID NO: 8), preferably at least 80% identity to ETV2 (SEQ ID NO: 1), more preferably at least 90% identity to ETV2 (SEQ ID NO:1), preferably at least 95% % identity to ETV2 (SEQ ID NO: 8), alternatively at least 98% identity to SEQ ID NO:1, alternatively 99% sequence identity to SEQ ID NO: 8, alternatively 100% sequence identity to SEQ ID NO:8.

After initiating transient expression of ETV2 in the PSCs, these cells were cultured for a sufficient time to produce a population of ETV2-hemogenic endothelial cells (ETV2-HECs). In particular embodiments, such a sufficient time is a period of about 24 hours to about 4 days. In some embodiments, a sufficient amount of time to produce a population of ETV2-hemogenic endothelial cells comprises culturing the ETV2-cells for about 1-2 days. In some embodiments, a sufficient amount of time to produce a population of ETV2-hemogenic endothelial cells comprises culturing the ETV2-cells for about 3-8 days, for example, for about 4 days. For example, in some embodiments, the step to produce a population of ETV2-hemogenic endothelial cells comprises culturing cells for 3 days, alternatively 4 days, alternatively 5 days, alternatively 6 days, alternatively 7 days, alternatively 8 days to produce ETV2-hemogenic endothelial cells but at a minimum at least 24 hours.

In some embodiments, the culture medium was “serum-free” which refers to cell culture materials that are free of serum obtained from animal (e.g., fetal bovine) or human blood.

In some embodiments, the culture conditions are feeder-free, meaning that the conditions do not use feeder cells. In particular embodiments, the culture conditions are serum-free and feeder-free.

In particular embodiments, the methods disclosed herein use a maintenance culture medium for culturing the PSCs after transfection with ETV2 mmRNA. In particular embodiments, the culture medium is E8-TeSR (commercially available from Stem Cell Technologies). The terms “E8 culture medium” and “E8” are used interchangeably and refer to the chemically defined culture medium having the following defined components: DMEM/F12, L-ascorbic acid-2-phosphate magnesium, sodium selenium, and NaHCO₃ in a final volume of 200, further comprising transferrin, insulin, FGF2 and TGFβ1.

In some embodiments, the culture medium is a xeno-free cell culture medium. In some embodiments, the culture conditions are xeno-free, serum free and feeder-free.

Serum-free and xeno-free medium suitable for culturing the ETV2-hemogenic endothelial cells to produce myeloid progenitors are known in the art and include, but are not limited to, for example, serum-free hematopoietic stem cell expansion medium StemLine II (commercially available from Sigma Aldrich, catalogue number S0189).

In some embodiments, a sufficient amount of time for culturing the ETV2-HECs to produce myeloid progenitors is at least 4 days, for example, at least 4-23 days. In some embodiments, a sufficient amount of time for culturing the ETV2-HECs to myeloid progenitors is about 7 to 12 days.

In some embodiments, the methods disclosed herein comprise isolating the myeloid cells from the culture. Suitable methods of isolating the cells are known in the art. In one example, non-adherent cells can be collected from the culture leaving the adherent cells behind. In some embodiments, the adherent cells isolated from the non-adherent myeloid cells, can be used in methods of producing neutrophils.

For production of neutrophils, the methods provided herein comprise the step of culturing the myeloid progenitors in a culture medium comprising granulocyte-colony stimulating factor (G-CSF) and a retinoic acid receptor agonist for a sufficient time to differentiate the myeloid progenitors into modified neutrophils. In some embodiments the retinoic acid receptor agonist is AM580, a retinoic acid receptor agonist (Stem Cell #72964) having a structure identified as CAS No. 102121-60-8.

A suitable time to differentiate the myeloid progenitors into modified neutrophils includes for at least 6 days, for example at least 6-21 days. In some embodiments, a suitable time for culturing the myeloid progenitors to differentiate into modified neutrophils is about 6 to 8 days.

Serum-free and xeno-free medium suitable for culturing the myeloid progenitors to differentiate into modified neutrophils are known in the art and include, but are not limited to, for example, StemSpan™ SFEM II (StemCell Technologies, Catalogue #09605 or 09655, stemcell.com/products/stemspan-sfem-ii.html) medium comprising Iscove's Modified Dulbecco's Medium, bovine serum albumin, recombinant human insulin, human transferrin (iron-saturated), 2-Mercaptoethanol, and other beneficial supplements.

In particular embodiments, the methods disclosed herein comprise selecting mature neutrophils from the population of neutrophils. In some embodiments, mature neutrophils are selected using a cell separation, cell sorting, or enrichment method, e.g., fluorescence activated cell sorting (FACS), enzyme-linked immunosorbent assay (ELISA), magnetic beads, magnetic activated cell sorting (MACS), and combinations thereof.

In particular embodiments, a mature neutrophil is identified by expression of cell surface markers. In particular embodiments, a mature neutrophil expresses CD15, CD101, and/or CD16.

Also provided herein is a population of modified mature neutrophils produced by the methods disclosed herein. The modified mature neutrophils have inhibited expression of MYDGF as well as superior anti-microbial and phagocytosis for therapeutic purposes. The properties of MYDGF KO induced neutrophils are described under the above section “2. MYDGF knock out (KO) induced neutrophils (iNeutrophils).”

While the methods disclosed herein include ordered, sequential events, the timing of the events can be varied by at least 20%. For example, while a particular step can be disclosed in one embodiment as lasting one day, the event can last for more or less than one day. For example, “one day” can include a period of about 18 to about 30 hours. Periods of time indicated that are multiple day periods can be multiples of “one day,” such as, for example, two days can span a period of about 36 to about 60 hours, and the like. In another embodiment, time variation can be lessened, for example, where day 2 is 48+/−3 hours from day 0; day 4 is 96+/−3 hours from day 0, and day 5 is 120 hours+/−3 hours from day 0.

### 4. Pharmaceutical Compositions

Provided herein are pharmaceutical compositions comprising a therapeutically effective amount of the population of neutrophils derived from pluripotent stem cells having inhibited expression of myeloid derived growth factor (MYDGF) described herein and a pharmaceutically acceptable carrier, wherein the neutrophils have enhanced anti-microbial function compared to neutrophils derived from pluripotent stem cells not having inhibited expression of MYDGF.

The term “therapeutically effective amount” refers to an effective dose or amount that is sufficient to elicit a beneficial or desired clinical result. The terms “pharmaceutical composition” or “therapeutic composition,” as used herein, refer to a compound or composition capable of inducing a desired therapeutic effect when properly administered to a subject. In some cases, pharmaceutical compositions appropriate for therapeutic applications can be in admixture with one or more pharmaceutically acceptable excipients, diluents, or carriers such as sterile water, physiological saline, glucose or the like. The terms “pharmaceutically acceptable carrier” or “physiologically acceptable carrier,” as used herein, refer to one or more formulation materials suitable for accomplishing or enhancing the delivery of the neutrophils of the disclosure.

### 5. Methods of Treatment

Provided herein are methods for treating a microbial infection in a subject comprising administering a pharmaceutical composition comprising a population of induced neutrophils having inhibited expression of MYDGF as described herein and a pharmaceutically acceptable carrier. In some embodiments, the microbial infection is a systemic infection. In some embodiments, the microbial infection is a fungal infection or a bacterial infection.

The terms “treatment” or “treat,” as used herein, refer to both therapeutic treatment and prophylactic or preventative measures. Those in need of treatment include subjects having microbial infection. In some embodiments, the methods, pharmaceutical compositions, and combinations disclosed herein can be used for the treatment of microbial infection.

The term “subject” is intended to include human and non-human animals, particularly mammals. In certain embodiments, the subject is a human patient.

The terms “administration” or “administering,” as used herein, refer to providing, contacting, and/or delivering a compound or compounds by any appropriate route to achieve the desired effect. Administration can include, but is not limited to, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, or via inhalation.

The term “microbial infection” refers to penetration and manifestation of microorganisms such as viruses, bacteria, fungi, or other microbes in the body. This can lead to cellular damages and symptoms of illness.

Groups of patients that are benefited from treatment with MYDGF KO neutrophils can include neutropenic patients (neutrophil deficiency), autoimmune diseases, and patients undergoing radiation/chemotherapy treatment (neutrophil depleted).

Without limiting the disclosure, a number of embodiments of the disclosure are described below for purpose of illustration. For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Materials and Methods

Human iPSC Culture and iNeutrophil Differentiation

Bone marrow-derived human iPSCs (hiPSCs)—IISH2i-BM9 (WiCell)—were differentiated into neutrophils as previously described in Brok-Volchanskaya et al., 2019, Stem Cell Reports 13 (6): 1099-1110 and U.S. Patent Publication No. US20200385676A1, incorporated by reference herein. hiPSCs were cultured in mTesR-Plus medium (STEMCELL Technologies) on tissue culture plates coated with Cultrex™ (Biotechne).

hiPSCs were first differentiated into hemogenic endothelium in TeSR-E8 medium (STEMCELL Technologies) using ETS Variant Transcription Factor 2 (ETV2) modified messenger RNA (mmRNA) (obtained from TriLink Biotechnologies), TasnIT reagent and mRNA boost (obtained from Mirus Bio). Cells were detached using TrypLE Select (Life Technologies) one hour prior to transfection. Lifted cells were then plated onto tissue culture plates coated with human collagen type IV (2.4 μg/mL; obtained from Millipore Sigma) in TeSR-E8 medium supplemented with ROCK inhibitor Y-27632 (ROCKi; obtained from Tocris). One day after transfection, the medium was modified to StemLineII media (obtained from Sigma Aldrich) supplemented with fibroblast growth factor 2 (FGF2) (20 ng/mL; obtained from PeproTech) and vascular endothelial growth factor VEGF-165 (20 ng/ml; obtained from PeproTech) to induce hemogenic endothelial cell differentiation; this cocktail is known as Media A. After two days media was changed to induce granulocyte-monocyte progenitor differentiation using StemLineII media (obtained from Sigma Aldrich) supplemented with FGF2 (20 ng/ml; obtained from PeproTech), granulocyte-macrophage colony-stimulating factor (GM-CSF) (25 ng/ml; obtained from Peprotech), and UM171 (50 nM; obtained from Xcess Biosciences); this cocktail is known as Media B. On days 8 to 10 following transfection, suspension cells were collected and plated for terminal neutrophil differentiation on tissue culture treated plates. These cells were cultured using StemSpan SFEM II medium (obtained from STEMCELL Technologies) supplemented with ExCyte 0.2% (obtained from Merck Millipore), human granulocyte colony-stimulating factor (G-CSF) (150 ng/ml; obtained from Amgen), GlutaMAX (1×; obtained from Thermo Fisher Scientific), and AM580 (2.5 μM; obtained from STEMCELL Technologies) at a density of 1×10⁶ cells/mL; this cocktail is known as Media C. After four days, Media C was refreshed on the suspension cells. iNeutrophils were then collected from the supernatant 8 to 10 days after this media change.

### Generation of MYDGF^{-/-} (MYDGF KO) BM9-iPSCs

To successfully generate MYDGF-KO iPSCs, two single guide RNAs (sgRNAs) were designed with Synthego's CRISPR design tool for targeting human MYDGF exon 4: 5′-CAAGAUACUCACGUAGGCC-3′ (sgRNA1) (SEQ ID NO: 1) and 5′-UCUGUACUUCACACAGUUCA-3′ (sgRNA2) (SEQ ID NO: 2). Other sgRNAs can be designed to target exon 4 of MYDGF in order to knock out (KO) expression and such designs are within the ability of a skilled person in the art. Before nucleofection, BM9 iPSCs were detached using TrypLE Select (obtained from Life Technologies) and singularized. Then 5 μg of each sgRNA and 5 μg of Cas9 protein (obtained from PNA Bio) were incubated for 10 minutes at room temperature. Cells were nucleofected using the Human Stem Cell Nucleofector Kit 2 (#VPH-5022; obtained from Lonza). Cells were then plated at 25 cell/cm² in mTeSR-Plus media with CloneR supplement (1×; obtained from STEMCELL Technologies) on a Cultrex-coated tissue culture plate. Two days following nucleofection, the media was changed to mTeSR-Plus only. Individual cell colonies were picked after seven days and expanded further.

To confirm the desired MYDGF mutation had been achieved in these cells, genomic DNA was extracted from each clone and screened using polymerase chain reaction (PCR) for acquisition of a 42 base pair deletion using primer 1 (5′-AATGGCGTCCATGTTGAGAAGC-3′) (SEQ ID NO: 3), primer 2 (5′-GGGCGCTGAGATTGAGTACG-3′) (SEQ ID NO: 4), and primer 3 (5′-TTCCTGCAAGTTACACACCCTACC-3′) (SEQ ID NO: 5) (all primers from Integrated DNA Technologies). Following Western blot confirmation and functional analysis of multiple MYDGF/clones, one clone (#6) was selected for experimentation. Other clones were characterized to confirm similarity in surface markers and some functional outputs.

### Flow Cytometry

Flow cytometry analysis was performed to investigate expression of cell lineage markers present on iNeutrophils. iNeutrophils were stained in Dulbecco's Phosphate Buffered Saline (DPBS)+1% human serum albumin (HSA)+Brilliant Buffer (obtained from Thermo Fisher Scientific) and fixed with 4% paraformaldehyde (PFA) (16%; obtained from Thermo Fisher Scientific). Sample acquisition and data collection were performed on an Aurora Cytometer (obtained from CytekBio). All antibodies utilized in this study are outlined in Table 1. Single cells were identified using forward and side scatter parameters and live cells identified using Zombie NIR dye (100 test; obtained from Thermo Fisher Scientific). The myeloid cell population was identified using CD11b expression and mature neutrophils were identified using CD15/CD16 expression. Monocytes were identified using CD14 and excluded from results. All data were analyzed with FlowJo Software (v10.8.1; obtained from TreeStar).

TABLE 1
Antibodies used in Flow Cytometry experiments.
Flow Cytometry Antibodies

| Target | Fluor | Clone | Cat # | Vendor |
|---|---|---|---|---|
| CD15 | BV711 | W6D3 | 563142 | BD biosciences |
| TLR2 | PE | 11G7 | 565349 | BD biosciences |
| CD11b | PE-Cy7 | ICRF44 | 301321 | Biolegend |
| CD15 | APC-Fire 810 | W6D3 | 323058 | Biolegend |
| CD16 | AF700 | 3G8 | 302026 | Biolegend |
| CD11b | PE | M1/70 | 101208 | Biolegend |
| CD15 | APC | W6D3 | 323008 | Biolegend |
| CD16 | BV711 | 3G8 | 563127 | BD biosciences |
| TLR2 | FITC | W15145C | 392307 | Biolegend |
| TLR4 | PE | HTA125 | 312806 | Biolegend |
| CD11b | BV605 | M1/70 | 101057 | Biolegend |
| CD16 | PE-Cy7 | 3G8 | 557744 | BD biosciences |

### Fluorescence-Based Reactive Oxygen Species (ROS) Assay

A plate reader, fluorescent signal-based assay was used to evaluate production of intracellular reactive oxygen species (ROS) by the iNeutrophil cell lines. One day prior to experimentation, a 96-well black, clear-bottom tissue culture treated plate was coated with 1× fibronectin bovine plasma (100×; obtained from Sigma-Aldrich) and incubated at 4° C. overnight. On the day of experimentation, cells were pelleted and resuspended in RPMI 1640 (no phenol red; obtained from Thermo Fisher Scientific)+2% fetal bovine serum (FBS) (obtained from R&D Systems). Dihydrorhodamine (DHR) 123 (5 mM; obtained from Thermo Fisher Scientific) was added to the resuspended cells at a 1:2000 dilution. Cells were then plated at 1×10⁵ cells per well and incubated for 30 minutes at 37° C., 5% CO₂. During the incubation phorbol myristate acetate (PMA) (obtained from Sigma-Aldrich) and Zymosan (obtained from Invivogen) stimuli were prepared at a two-fold (2×) concentration, so that when added to wells they were at 100 ng/mL and 100 μg/mL, respectively. Stimuli or media (control) were added to appropriate wells following incubation. The fluorescent signals were detected at 485/535 nm in a microplate reader (Synergy H1; obtained from Bio-Tek Instruments). Background fluorescence of the media-only control wells was subtracted from each sample prior to data analysis.

### Immunoblotting

Western blotting was performed to quantify MYDGF protein expression and assess phospho- and total-protein signaling in iNeutrophil lines. To promote activation, cells were stimulated with fMLP (1 μM; obtained from Sigma-Aldrich). Cells were pelleted and pellets collected in lysis buffer. Lysis buffer was made up of HEPES (25 mM, pH 7.5), NaCl (150 mM), Nonidet P-40 (1%), MgCl₂ (10 mM), EDTA (1 mM), glycerol (10%) supplemented with pepstatin A (1 g/mL), aprotinin (2 g/mL), leupeptin (1 g/mL), phenylmethanesulfonyl fluoride (200 nM), sodium orthovanadate (1 mM), protease inhibitor mixture 2 (obtained from Sigma-Aldrich). The cells were then incubated on ice for 10 minutes and sonicated for 3×5 seconds at 20% amplitude. Next, cells were clarified using centrifugation (15,000 g, 4° C. for 10 minutes). Protein concentrations were then evaluated using the BCA Protein Assay (obtained from Thermo Fisher Scientific) and all samples were stored at −80° C. Cell lysates were boiled with 1×SDS running buffer (20×; Thermo Fisher Scientific) and immunoblotting was performed. Blots were imaged using an infrared imaging system (Odyssey; obtained from LI-COR Biosciences). All primary and secondary antibodies used for blotting can be found in Table 2.

TABLE 2
Antibodies used in Western Blot experiments.
Western Blot Antibodies

| Target | Primary/Secondary? | Cat # | Vendor |
|---|---|---|---|
| anti-Rabbit 800 | Secondary | 926-32211 | Licor |
| anti-Mouse 680 | Secondary | A21057 | Invitrogen |
| Beta-tubulin (total) | Primary | 4585 | Sigma |
| MYDGF (total)* | Primary | — | — |

*MYDGF Western blot antibody is described in Bortnov et al., The Journal of biological chemistry 293(34): 13166-13175, (2018).

### Cytospins

Cytospin experiments were performed to confirm iNeutrophil morphology and assess cell clustering. In these experiments, 80,000 cells were spun onto glass slides at 1200 rpm/200 g for 5 minutes using the Shandon Cytospin 3 centrifuge (obtained from Thermo Fisher Scientific). Slides were stained with Differential Quick (Diffquik) III Stain Kit (obtained from Electron Microscopy Sciences) following the manufacturer's protocol. Slides were imaged using 20× magnification on an Olympus IX70 microscope. iNeutrophil morphology was evaluated by checking for hyper-segmented nuclei and cluster sizes were evaluated using the ImageJ polygon tool and measurement tool for calculating area.

iPSC-Derived Endothelial Cell Differentiation and Culture

iPSC-derived endothelial cells were generated using ETV2 transfection and differentiating wild-type IISH2i-BM9 iPSCs (WiCell) through the hemogenic endothelium stage as previously described (Peterson et al., J Cell Sci. 137 (3): jcs261774, (2024)). The IISH2i-BM9 iPSCs were transfected with ETV2 mmRNA and cultured in StemLineII media (obtained from Sigma-Aldrich) supplemented with FGF2 (20 ng/ml; obtained from Peprotech) and VEGF-165 (20 ng/ml; obtained from PeproTech). Two days following transfection, medium was changed to Vasculife Basal Medium (obtained from LifeLine Cell Technologies) supplemented with the iCell-Endothelial Cells Medium Supplement (obtained from CDI). iPSC-derived endothelial cells were plated in tissue culture flasks pre-incubated with 1× fibronectin bovine plasma (100×; obtained from Sigma-Aldrich).

### LumeNEXT Device Preparation

All LumeNEXT devices were fabricated as previously described (Jiménez-Torres et al., Adv Healthc Mater. 5 (2): 198-204, (2016)). Devices were formed by patterning 2-polydimethylsiloxane (PDMS) layers created from SU-8 silicon master molds (obtained from MicroChem). These devices were then bonded onto glass-bottom MatTek dishes using oxygen plasma (Diener Electronic Femto Plasma Surface System) and a PDMS rod was threaded through the device chamber. The LumeNEXT devices were assembled as previously described (Jiménez-Torres et al., 2016, Id.). Specifically, collagen I (obtained from Corning) was neutralized using NaOH to pH7.4 (3 mg/mL) and then pipetted into the devices so as to form around the PDMS rods. Following collagen polymerization, PDMA rods were removed from the devices, leaving behind a lumen channel. The lumens were then seeded with iPSC-derived endothelial cells at a density of 2.5×10⁴ cells/μL. The lumens were flipped in the incubator (37° C.) every 10 minutes for 30 minutes to ensure appropriate endothelial cell distribution around the channel. After the flips were complete, non-adhered cells were aspirated and replaced with fresh endothelial media. The lumen devices were cultured for two days with regular medium changes.

### Trans-Endothelial Migration Assay

Endothelial lumens were prepared using previously published LumeNEXT protocols; see, Hind et al., Blood, 132 (17), 1818-1828, (2018); Peterson et al., Journal of cell science, 137 (3), jcs261774, (2024). Following the two days of lumen culture and medium changes, a trans-endothelial migration assay was run to determine capacity of iNeutrophils to migrate across a physiologically-relevant barrier. Cells were first stained with Calcein AM (1:1000 in DPBS; obtained from Fisher Scientific) for 15 minutes at 37° C. After staining, cells were resuspended in endothelial media at a density of 7.5×10⁶ cells/mL and placed into the lumen channels. The top port of the lumen devices received either endothelial media as a control or Pseudomonas aeruginosa to induce migration. Migration was tracked over the course of 24 hours and movies were analyzed using ImageJ.

### Metabolomics

Abundance of intracellular metabolites was assessed following exposure of the iNeutrophils to zymosan (100 μg/mL). Approximately 2×10⁶ iNeutrophils were collected and pelleted in a 1.5 mL microcentrifuge tube. The cells were then washed once with PBS (1×) and then resuspended in 500 μL of StemSpan SFEM II medium supplemented with GlutaMAX (100×), ExCyte (0.2%), human granulocyte colony stimulating factor (GCSF) (150 ng/ml), and AM580 (2.5 μM). After resuspension in this media, 500 μL of zymosan supplemented media was added to the experimental condition tubes. Media not containing zymosan was added to other tubes to act as unstimulated controls. Tubes were left to incubate for 2 hours at 37° C.+5% CO₂. Gentle pipetting was performed every 40 minutes during the incubation period to mix the cells and ensure equal exposure to zymosan. After the incubation, cells were then pelleted and the supernatant was removed. The cell pellet was washed with 1 mL of PBS (1×). Next, 500 μL of cold MeOH:H₂O (80:20) solution was added to each of the tubes which were then vortexed and placed at −80° C. for 30 minutes. Once removed from the freezer, tubes were vortexed and spun down (13,000 rpm for 5 minutes, 4° C.). The supernatant was collected into a fresh microcentrifuge tube over dry ice. An additional 150 μL of the cold MeOH:H₂O solution was added to the tubes with the original cell pellets. These tubes were once again vortexed and centrifuged and the subsequent supernatants were transferred to their corresponding tubes on dry ice. For each experimental condition, there were three technical replicates which were extracted for analysis.

Intracellular metabolites were measured using a Thermo Q-Exactive Mass Spectrometer paired with a Vanquish Horizon UHPLC. The data were collected using Xcalibur 4.0 Software and peaks were integrated using Maven Software. Data was collected on full scan negative mode and measurements of identified metabolites were based on m/z and retention times determined using chemical standards. Metabolite extracts were dried under nitrogen stream following their storage in −80° C. MeOH:H₂O. The samples were then resuspended in LCMS-grade water and separated using a 2.1×10⁰ mm, 1.7 μM Acquity UPLC BEH C18 Column.

### Inflammatory Cytokines Enzyme-Linked Immunosorbent Assays (ELISAs)

ELISA was used to determine the concentrations of secreted inflammatory cytokines IL-8, IL-1β, IL-6 and TNFα by iNeutrophil cell lines. A tissue culture-treated 12-well plate was coated with fibronectin (10 ug/mL). The cells were resuspended in Phenol Red Free RPMI+2% FBS and 1.5 million iNeutrophils were plate per well of the 12-well plate. The cells were left to rest for 30 minutes at 37° C. prior to stimulation with either E. coli LPS (200 ng/mL) or zymosan (10 μg/mL) for 4 hours at 37° C. iNeutrophils were collected and spun down at 350 g. All supernatants were collected and frozen at −80° C. Cytokines concentrations were assessed using Human IL-8/CXCL8 DuoSet ELISA (Bio-Techne), Human IL-1 beta/IL 1F2 DuoSet ELISA (Bio-Techne), Human IL-6 DuoSet ELISA (Bio-Techne), and Human TNFα DuoSet ELISA (Bio-Techne) following manufacturer instructions. All supernatant samples were appropriately diluted to fit within the standard curve and normalized to cell input number.

Aspergillus fumigatus Co-Culture Assay and Imaging

Red fluorescent protein (RFP)-expressing Aspergillus fumigatus (CEA10) was grown as previously described (Schoen et al., PLOS pathogens 19 (5), e1011152, (2023)). Briefly, A. fumigatus was grown on glucose minimal medium (GMM) plates in the dark at 37° C. to induce asexual conidiation. A. fumigatus was plated at 1×10⁶ conidia/10 cm plate for 3 days. The conidia were collected in water+0.1% Tween by scraping with an L-spreader and subsequent passage through 0.4 μm Miracloth into a 50 ml conical tube. A 5 mL aliquot of this spore suspension was transferred to a new tube and then centrifuged for 5 min at 800 g and resuspended in 50 mL of PBS (1×). The collected conidia were the counted using a hemacytometer and the final concentration was adjusted to 4×10³ spores/mL.

Following spore collection, 1 mL of a 4×10³ suspension was added to wells of a 24-well plate coated with fibrinogen (10 μg/mL). This plate was incubated for 8 hours at 37° C. until germlings were visible under a microscope. iNeutrophils were collected and diluted to a concentration of 8×10⁵ cells/mL. Once germlings developed, glucose minimal medium (GMM) was removed from all wells and 500 μL of iNeutrophil suspensions were plated to yield a final neutrophil: germline ratio of 100:1. Images were taken every 3 minutes for 12 hours on a Nikon Eclipse TE300 inverted fluorescent microscope using the 20× objective. The microscope environmental chamber was set to 37° C. with 5% CO₂. Videos were compiled with NIS Element and ImageJ software. Fiji ImageJ was used to quantify hyphal growth. Fungal size was measured for all viable fungi at time 0 (start) and time 12 (end) to assess growth over time.

### Assay for Oxidative Burst/Oxygen Consumption Rate Assay

Oxidative burst in response to stimuli was evaluated using the Seahorse metabolic Mito Stress Test Assay (Agilent) on the Seahorse XFe96 Analyzer (both obtained from Agilent). One day prior to performing this assay, the Seahorse cell culture microplate (obtained from Agilent) was coated with 1× fibronectin bovine plasma (100×; obtained from Sigma-Aldrich) and incubated at 4° C. overnight. Also one day prior to the assay, the Seahorse sensor cartridge (obtained from Agilent) was hydrated with endotoxin-free water (<0.005 EU/mL; obtained from Quality Biological) and incubated at 37° C. On the day of experimentation, the water in the cartridge was replaced with Seahorse XF Calibrant (obtained from Agilent). Cells were plated on the cell culture microplate at a density of 4×10⁴ cells/well. PMA (2,000 nM; obtained from Sigma-Aldrich) was placed in port A of the Seahorse sensor cartridge for a final concentration of 200 nM. The Mito Stress Test Assay template was modified on the Seahorse Analyzer to include only port A injection and measured for 2 hours following baseline measurement. All data were analyzed using Wave Analyzer (obtained from Agilent) and Prism (v9; GraphPad).

### Single Cell RNA Sequencing

2×10⁵ iNeutrophils were collected at day 7 following transfer into Media C, spun down, and resuspended in 200 μL of PBS+0.04% Bovine Serum Albumin (BSA; Sigma-Aldrich). All cells were directly used for library construction in a Chromium controller at the Gene Expression Center of the University of Wisconsin-Madison Biotechnology Center (RRID: SCR_017757). Libraries were constructed according to the Chromium Single Cell 3′ Reagent Kit v3.1 user guide (10× Genomics, Pleasanton, CA). Cell concentration and viability were quantified on the Luna-FX7 (Logos Biosystems, Anyang, South Korea) using acridine orange/propidium iodide stain. Cells were loaded onto a Chromium Single Cell Chip G chip for the single cell partitioning GEM reaction on the Chromium iX platform. Next, GEMs were transferred to emulsion safe strip tubes for reverse transcription and cell/transcript barcoding using an Eppendorf MasterCycler Pro thermocycler (Eppendorf, Hamburg, Germany). Following reverse transcription, GEMs were broken, and the pooled single cell cDNA was amplified for 13 cycles and purified using SPRIselect beads (Beckman Coulter, Brea, CA) and quantified on a Bioanalyzer 2100 (Agilent, Santa Clara, CA) using the High Sensitivity DNA kit. A portion of the full-length cDNA was fragmented and used to generate cDNA libraries according to the standard 10X Genomics workflow. See supplementary materials for description of data processing, quality control, and data integration and annotations.

### Sequencing, Initial Data Processing and Quality Control

All libraries were pooled and sequenced at DNA Sequencing Facility (RRID: SCR_017759) on a NovaSeq 6000 sequencer S1 flow cell with paired-end, 50 bp sequencing. Data was processed with bcl2fastq for the generation of FASTQ files. Raw sequencing reads in the FASTQ files were mapped to GRCh38 human genome by Cell Ranger (v6.1.2, 10X genomics). Filtered cell-by-gene matrices were used for downstream analysis in R with Seurat (v4.3.0); see, Hao et al., Cell, 184 (13), 3573-3587.e29, (2021). Based on general distribution, only cells with more than 50 genes captured and a percentage of mitochondrial genes below 10% were pt. Doublets were labeled by DoubletFinder (v2.0.3), see McGinnis et al., Cell systems, 8 (4), 329-337.e4 (2019). with principal components (PCs) 1:30, and parameters for generated artificial doublets set at 25%, and neighborhood size at 0.09. Post doublet removal, each sample was normalized individually by SCTransform, regressing out the impact of number of features.

### Data Integration, Unsupervised Clustering and Population Annotations

To integrate normalized wildtype and MYDGF^{-/-} samples into one data object, the top 3000 highly variable genes in each sample were chosen as features for creating anchors. Samples were integrated by IntegrateData using the anchors as input, with normalization method set to SCT to match with SCTransform normalization process. Post integration, linear and nonlinear dimensional reduction and unsupervised clustering were performed. By examining the distribution of quality control measurements, one cluster that was low in number of features and did not have differentially highly expressed genes comparing to the rest was identified and removed. Post cluster removal, integration and dimensional reduction with principal component analysis (PCA) and UMAP were repeated. Specifically, PCs 1:30 were used in UMAP and neighbor-finding. Unsupervised clustering was done at a resolution of 0.1. For cluster annotations, differential expression analysis was done by Wilcoxon rank-sum test using FindAllMarkers with log 2 fold change threshold set to 0.25. Functional annotation for differentially expressed gene signatures was done by gene ontology enrichment analysis through Metascape (v3.5.20250101) and individual gene curation through literature review, see Zhou et al., Nature communications, 10 (1), 1523, (2019.

### Neutrophil Maturation Estimation

For maturation stage reference, the processed Seurat object provided by Montaldo et al. was used (Montaldo et al., Nature immunology, 23 (10), 1470-1483, (2022)). Briefly, SCTransform was performed for both the reference neutrophil atlas and iNeutrophil data as disclosed herein. Anchors between the two datasets were identified by FindTransferAnchors using PCA with PCs 1:30 as reference reduction space, and SCT as normalization approach. Label transfer from reference to iNeutrophils was performed by TransferData with the same dimensions used in anchor identification. Predicted maturation stage names were added to iNeutrophils as a new metadata.

### Underoil Migration System

Liquid-walled microchannels were constructed as previously described (Schrope et al., Advanced science (Weinheim, Baden-Wurttemberg, Germany) 12(21): e2414024, (2025)). Channels were coated with fibrinogen (10 μg/mL), fibronectin (1 μg/mL) and collagen IV (1 μg/mL) in RPMI+2% fetal bovine serum (FBS) for 30 minutes at 37° ° C., 5% CO₂. For experiments requiring quantification of intracellular calcium dynamics, cells were stained with Fluo4-AM fluorescent dye (1 μM) in RPMI media+2% FBS for 20 minutes at 37° C., 5% CO₂. For BAPTA AM experiments, iNeutrophils were pretreated for 30 minutes with BAPTA-AM (100 μM). After incubation of the channels, media was removed and 1.5 μL cells were seeded onto the inlet droplet at 60,000 cells/μL in RPMI media+2% FBS. Immediately after, 1.5 L of fMLP (10 μM) was seeded onto the outlet droplet and the channels moved to an on-stage incubator on a Nikon Eclipse TE300 inverted fluorescent microscope with a PanFluor 20×/0.50 NA objective or on Nikon Eclipse Ti2 microscope using Aprochromat 60×/1.42 NA or S-Fluor 20×/0.75 NA objectives in combination with Tokai Hit stage incubation system. For experiments tracking speed and/or fluo4 fluorescent intensity, images were acquired every 2.5 seconds for 5 minutes in GFP and brightfield channels. See Schrope et al., bioRxiv [Preprint]. 2024 Oct. 4:2023.06.14.544898. doi: 10.1101/2023.06.14.544898.

Single-cell quantification of calcium was achieved by measuring the Fluo4-AM intensity as a fold change in relation to time 0. Bulk quantification of calcium was achieved using MATLAB code provided in the supplement.

### Example 1

MYDGF Knockout (KO) iNeutrophils were More Immature, More Pro-Inflammatory, and More Efficient in Inhibiting Fungal Growth Compared to Wild-Type iNeutrophils

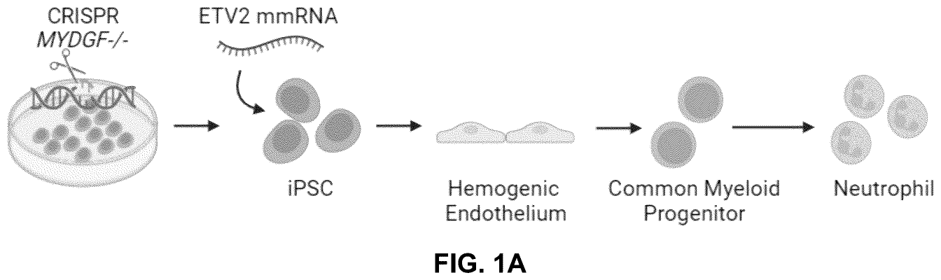

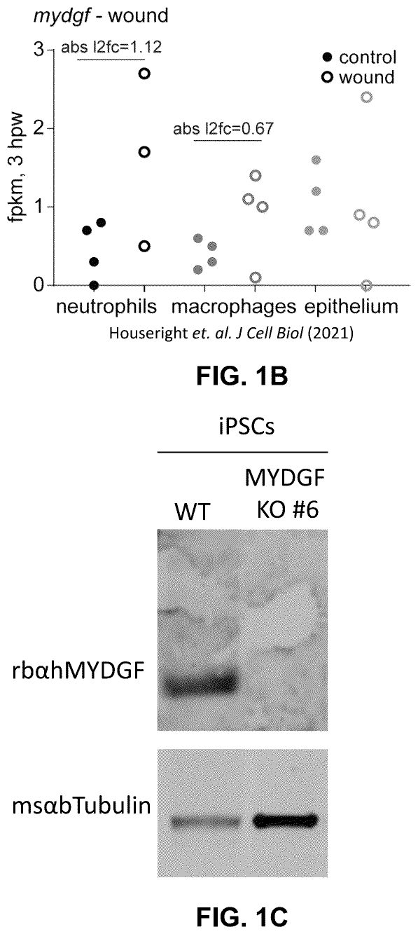

MYDGF expression had been shown to increase in zebrafish neutrophils in response to wound damage (shown in FIG. 1B) (Houseright et al., 2021, J Cell Biol. 220 (8): e202103054). It had also been shown that MYDGF regulated neutrophil responses to tissue damage but not infection (Houseright et al., Id.). As these findings were found from experiments performed in zebrafish's neutrophils only, experiments were set out to determine the role of MYDGF in induced neutrophils (iNeutrophils).

iNeutrophils were derived from bone marrow-derived human iPSCs (hiPSCs)-IISH2i-BM9 (WiCell) (shown in FIG. 1A) using the same method described in Brok-Volchanskaya et al., 2019, Stem Cell Reports 13 (6): 1099-1110 and U.S. Patent Publication US20200385676A1, expressly incorporated herein. CRISPR having sgRNAs targeting exon 4 of MYDGF was performed to knock out MYDGF expression. Deleted expression of MYDGF KO was confirmed by Western Blot (shown in FIG. 1C).

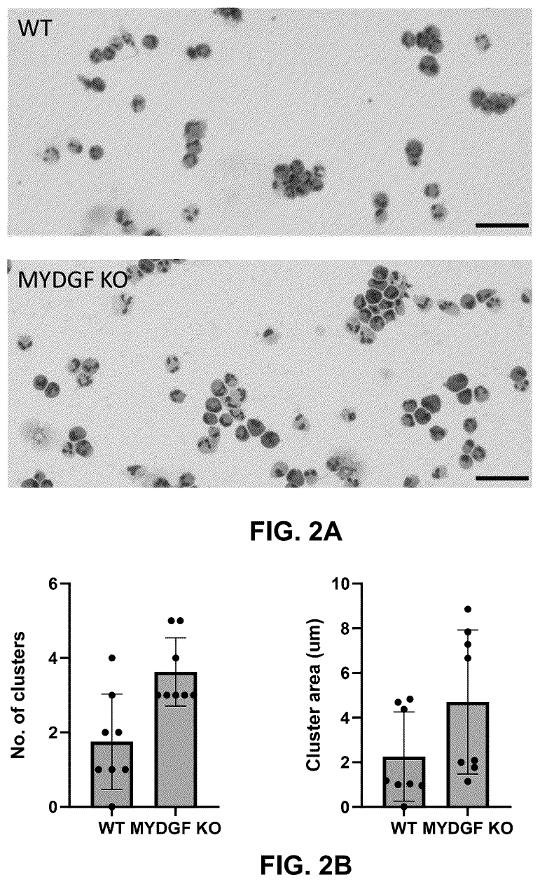

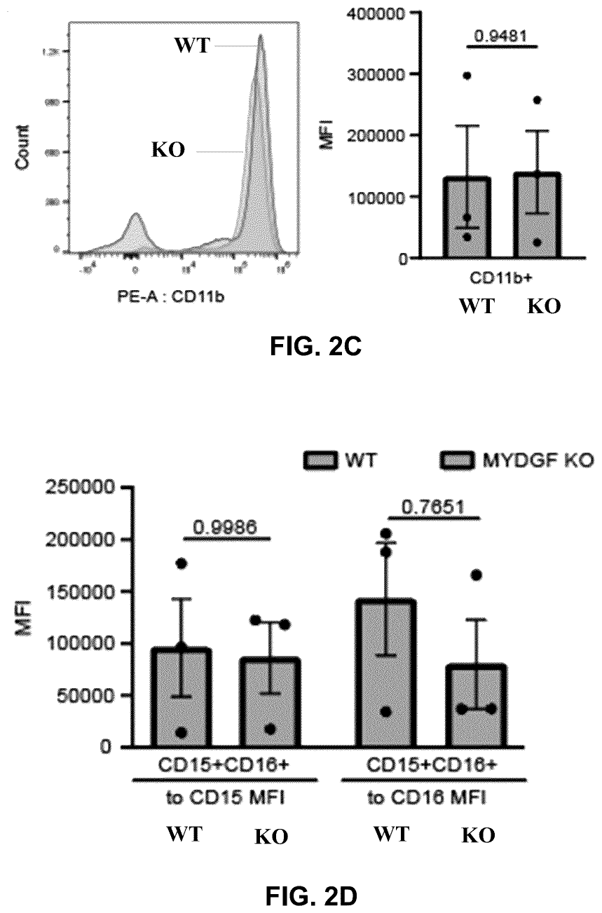

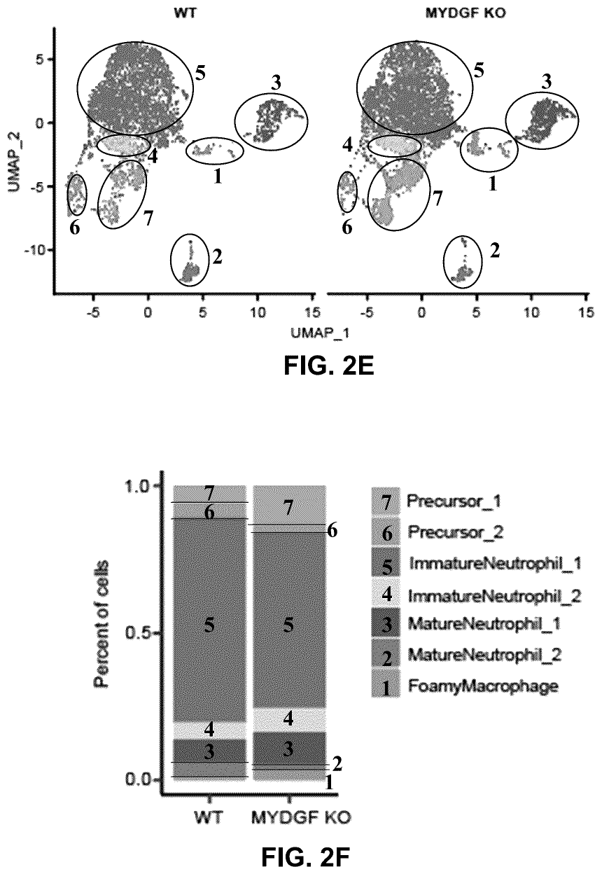

The morphology of wild type (WT) and MYDGF knockout (KO) iNeutrophils were first characterized by nuclei staining. MYDGF KO iNeutrophils tended to cluster more and in larger aggregates indicating more activation (as shown in FIG. 2A and FIG. 2B). However, there was no significant difference in the level of maturity to WT and KO cells, as indicated by the expression of the maturity related biomarkers (FIG. 2C and FIG. 2D). The single cell RNA sequencing data further confirmed that there were no differences in the types of cells within the iNeutrophil population of WT and MYDGF KO (FIG. 2E and FIG. 2F).

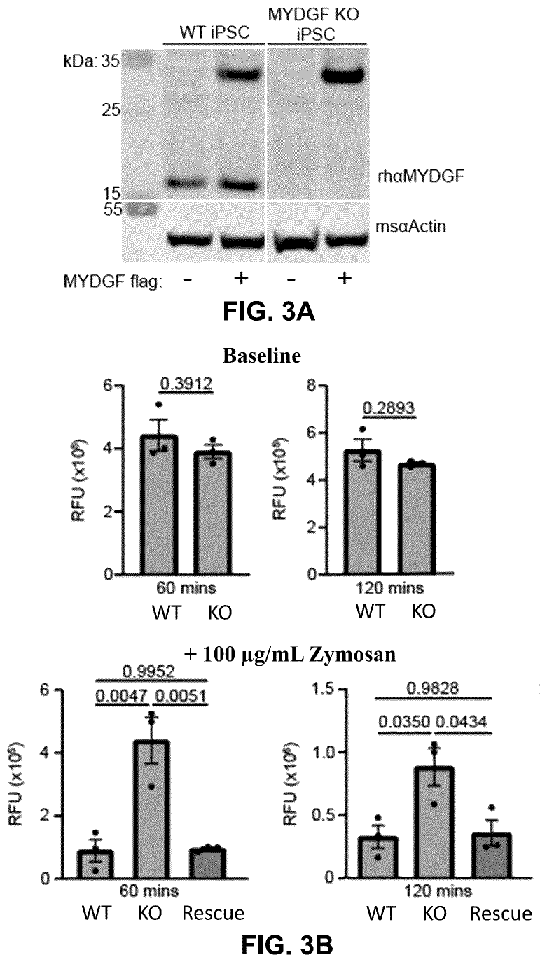

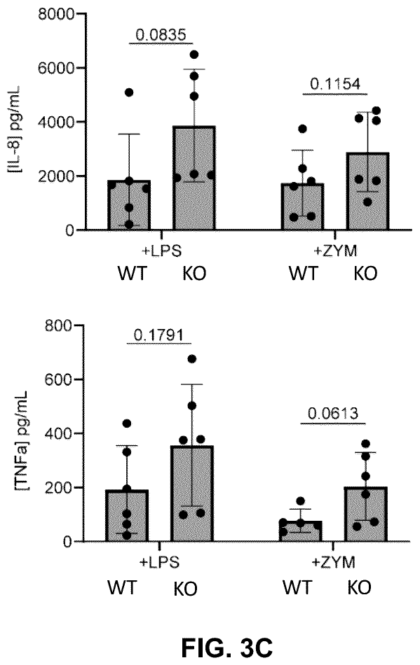

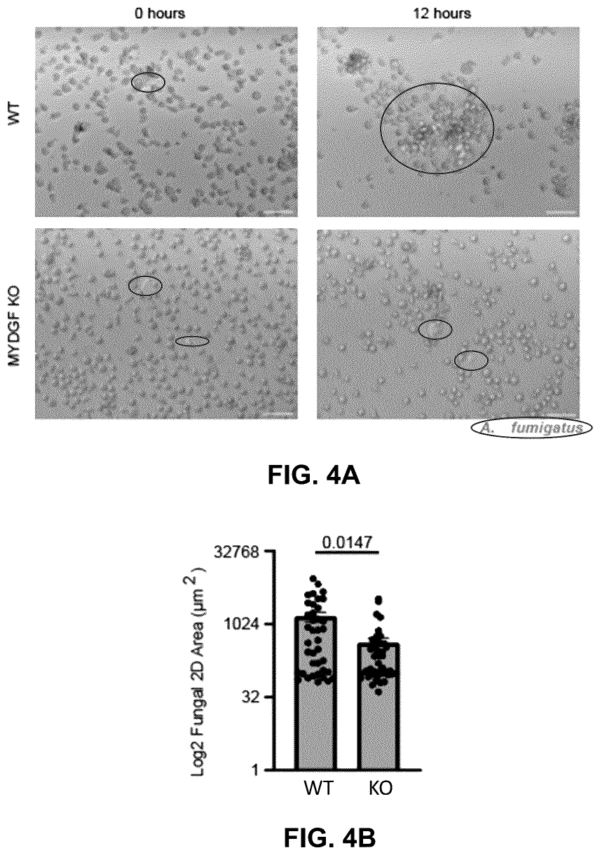

Neutrophils are key components of the innate response to bacterial infection. Particularly, neutrophils are known to produce reactive oxygen species (ROS) and cytokines to kill microbes in infection (Mayadas et al., Annu Rev Pathol. 9:181-218, (2014)). MYDGF KO iNeutrophils significantly produced more ROS compared to wild-type when stimulated by Zymosan (FIG. 3B). When the MYDGF expression was rescued in the MYDGF KO iNeutrophils, the ROS production was dampened, indicating that the ROS production level was influenced directly by MYDGP expression (FIG. 3A and FIG. 3B). Strikingly, MYDGF KO iNeutrophils were superior in inhibiting fungal growth after co-culturing with Aspergillus fumigatus (as shown in FIG. 4A and FIG. 4B).

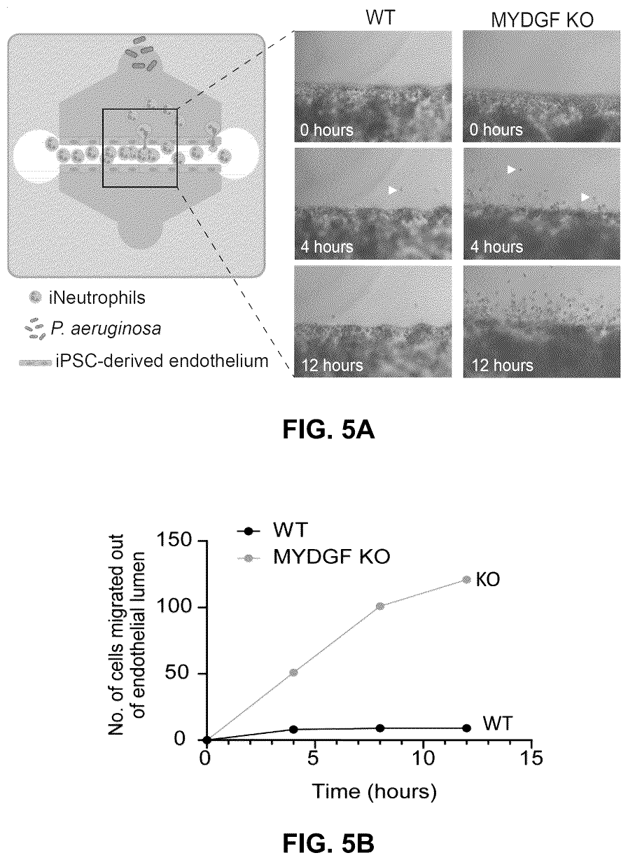

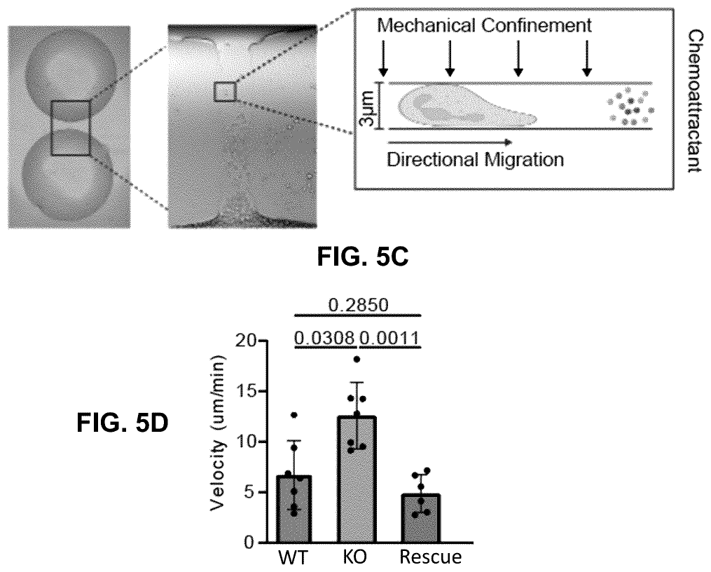

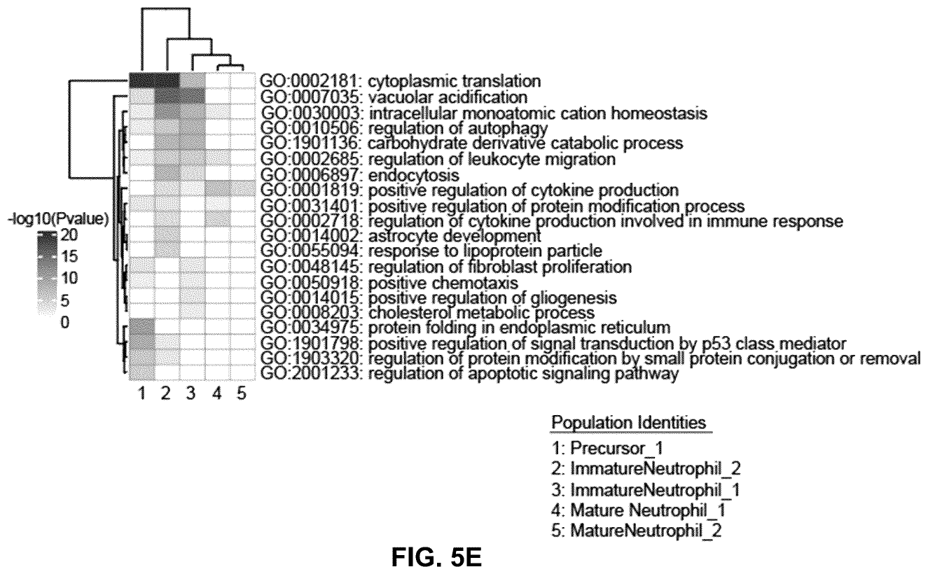

Additionally, MYDGF KO iNeutrophils had the tendency to secrete more pro-inflammatory cytokines (IL-8, IL-6, and TNFα) compared to wild-type (as shown in FIG. 3C). In a trans-endothelial lumen model of neutrophil migration in which iNeutrophils were sandwiched by layers of endothelial cells (shown in FIG. 5A), more KO iNeutrophils migrated in a given time across the endothelial barrier in response to Pseudomonas aeruginosa (shown in FIG. 5B). Similarly, in the confined model that mimics the small gaps between endothelial cells through which neutrophils have to move (FIG. 5C), the speed of the movement was higher in KO cells (FIG. 5D). This speed was dampened when MYDGF expression was rescued in the KO, indicating that MYDGF expression directly contributed to the superior mobility (FIG. 5D). Indeed, the single cell RNA sequencing data also identified top pathways regulated by the MYDGF KO iNeutrophils, some of which were directly associated with leukocyte migration (FIG. 5E). This data helps explaining how the MYDGF iNeutrophils were migrating faster and more efficiently to a stimulus.

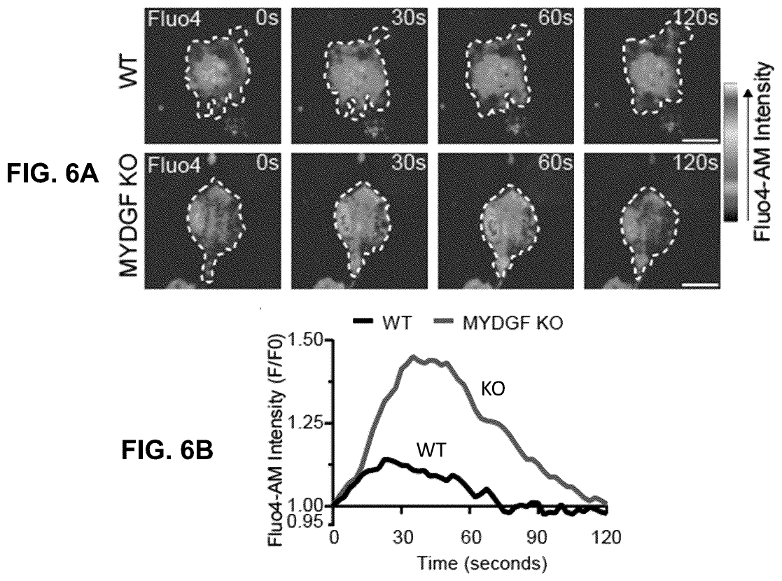

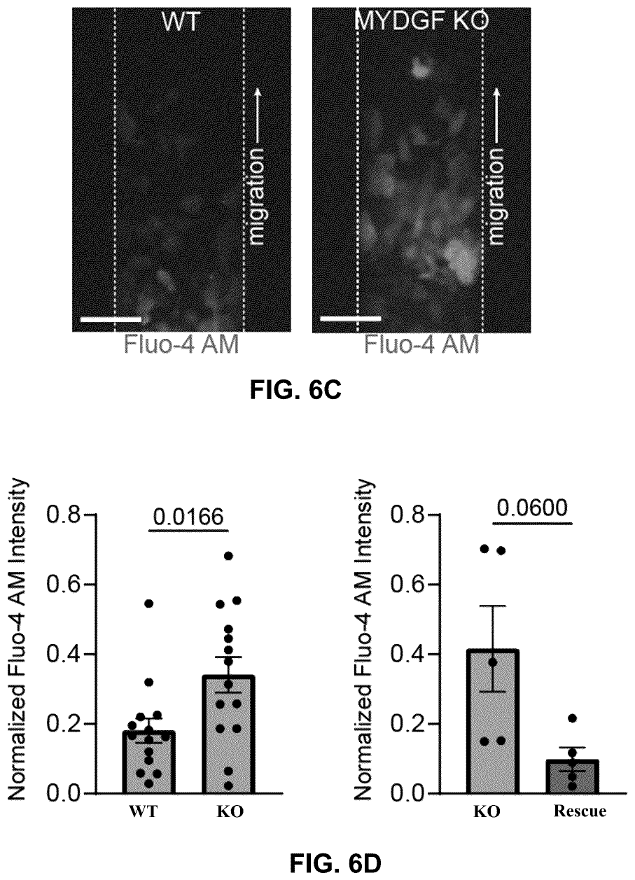

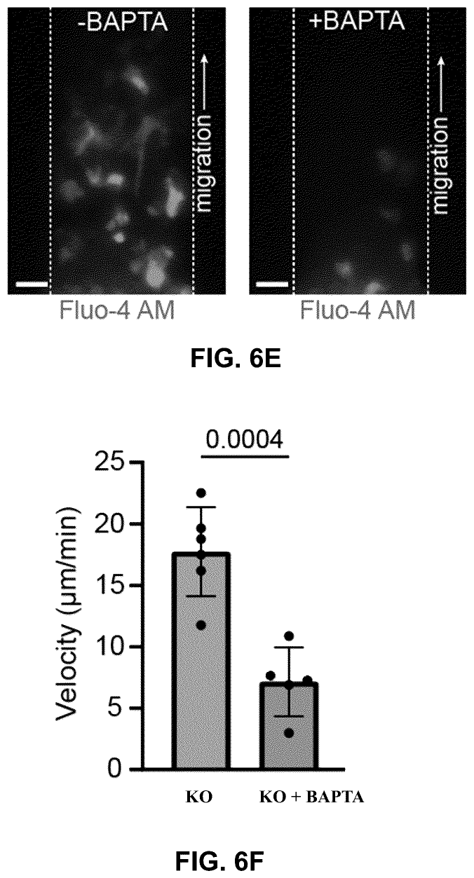

Calcium levels, which were an essential component in all neutrophil functions including ROS production and migration, was studied using Fluo4 calcium dye. Overall, calcium levels (measured by fluorescence intensity) and calcium transient events (measured by number of peaks per min) were higher in KO than in WT cells (FIGS. 6A-6E). MYDGF rescue in the KO returned calcium levels to that found in WT (FIG. 6F), suggesting direct role of MYDGF in regulating calcium dynamics in neutrophils and their functions.

These results demonstrated that MYDGF KO iNeutrophils were better activated, more robust in producing anti-microbial molecules such as ROS and pro-inflammatory cytokines. They were also more efficient in migrating towards infection and inhibiting fungal growth, and exhibited higher calcium level/events. Unexpectedly, these data suggested a role of MYDGF in regulating iNeutrophils in response to infection, which was in contrast with the previous findings in zebra fish's neutrophils (Houseright et al., 2021, J Cell Biol. 220 (8): e202103054).

### Example 2

MYDGF Regulated iNeutrophil-Mediated Response to Infection Through Toll-Like Receptor 2 (TLR2) and a Shift in Metabolism.

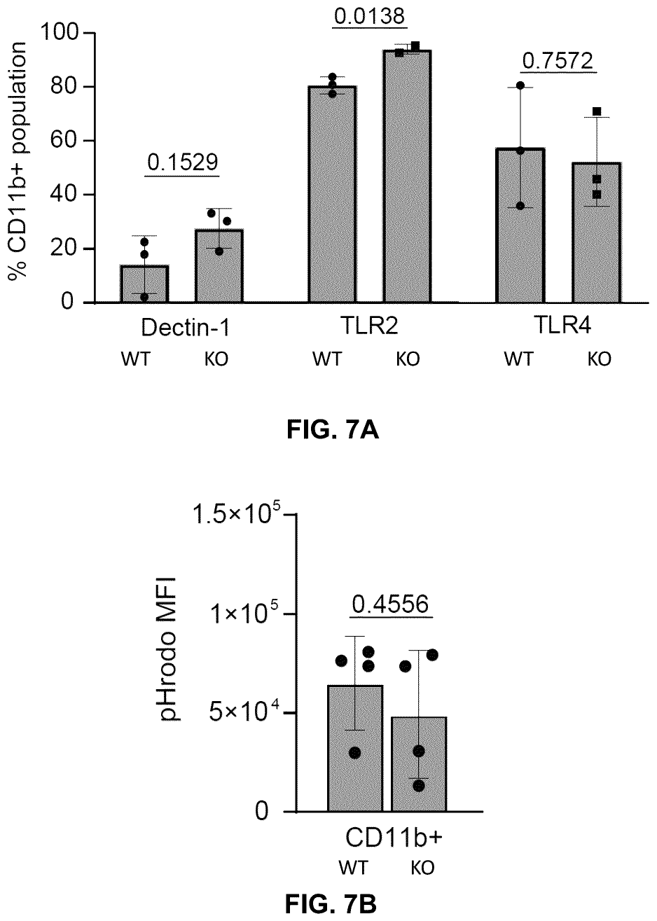

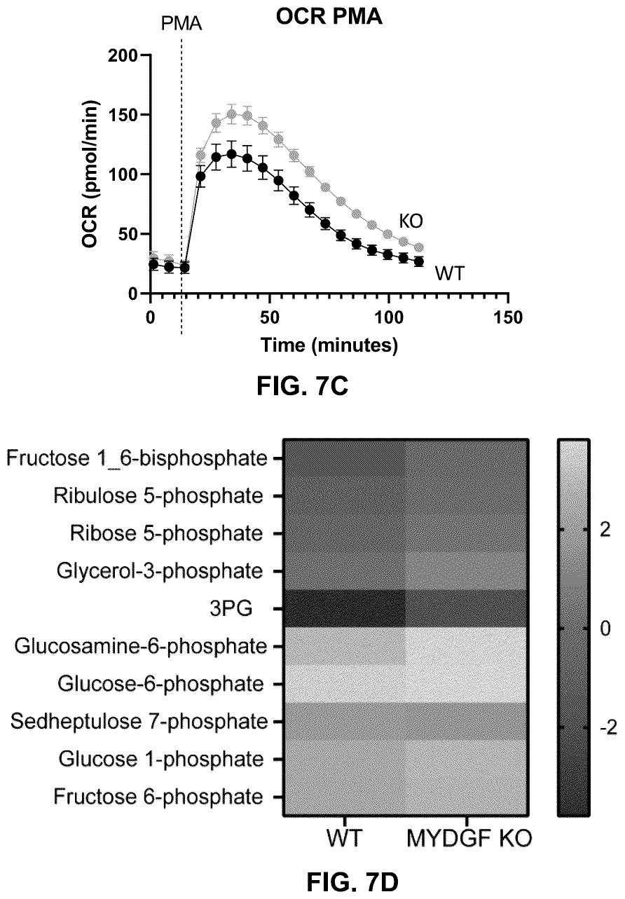

Experiments to investigate the mechanism through how MYDGF regulates iNeutrophil's function in response to infection were performed to assess pattern recognition receptors. Pattern recognition receptors (PRRs), including TLR2, Dectin-1 and TLR4, are receptors that detect general patterns and structures on potential pathogens and can trigger signaling cascade that activates ROS and cytokine production upon binding of pathological ligands (shown in FIG. 7A). MYDGF KO iNeutrophils expressed significantly more TLR2 and had a tendency to express more Dectin-1 and TLR4, compared to wild-type neutrophils (shown in FIG. 7B). However, phagocytic activity in KO iNeutrophils was not significantly different from that in wild-type, suggesting that direct phagocytosis was unlikely contributing to the enhanced antimicrobial property of the KO (shown in FIG. 7C). In addition, MYDGF KO iNeutrophils had a higher oxygen consumption rate (OCR) than wild-type, indicating a higher aerobic metabolism (shown in FIG. 7D). Consistent with these findings, expression of enzymes involved in glucose oxidative metabolism and anaerobic glycolysis was upregulated and downregulated respectively in MYDGF KO iNeutrophils compared to WT in response to zymosan (shown in FIG. 7E). An increase in glucose oxidative metabolism is consistent with a high demand of oxygen consumption during migration in these cells.

Taken together, these results demonstrated that MYDGF inhibited gene expression that supported oxidative glucose metabolism and regulated ROS and cytokine production. Release of these inhibitions in MYDGF KO iNeutrophils would support an increase in migration and activation in response to infection as observed in these cells.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

We claim:

1. A population of neutrophils derived from human pluripotent stem cells having inhibited expression of myeloid derived growth factor (MYDGF), wherein the neutrophils have enhanced anti-microbial function compared to neutrophils derived from pluripotent stem cells not having inhibited expression of MYDGF.

2. The population of claim 1, wherein the expression of MYDGF is inhibited by deletion of exon 4 of the MYDGF gene.

3. The population of claim 1, wherein expression of the MYDGF gene is inhibited using gene mutation, RNA-mediated inhibition, RNA editing, or DNA gene editing or base editing.

4. The population of claim 3, wherein the gene editing method comprises sequence modification using a nuclease selected from the group consisting of a meganuclease, ZFNs, TALENs, and Cas enzyme.

5. The population of claim 4, wherein the nuclease is a Cas9 enzyme.

6. A pharmaceutical composition comprising a therapeutically effective amount of the population of neutrophils having enhanced anti-microbial function of claim 1 and a pharmaceutically acceptable carrier.

7. A method of treating a microbial injection in a subject comprising administering the pharmaceutical composition of claim 6 to the subject.

8. The method of claim 7, wherein the microbial infection is a systemic infection, a bacterial infection, or a fungal infection.

9. (canceled)

10. The method of claim 6, wherein the composition is administered intravenously to the subject.

11. The method of claim 6, wherein the subject is a patient that has neutrophil deficiency, autoimmune disease, or neutrophil depletion due to radiation or chemotherapy treatment.

12. A method of producing neutrophils having inhibited expression of MYDGF from pluripotent stem cells (PSCs) comprising inhibiting expression of MYDGF in the PSCs and differentiating the PSCs into neutrophils.

13. The method of claim 12, wherein differentiating the PSCs into neutrophils comprises:
(a) transiently introducing exogenous ETV2 into the human pluripotent stem cells having inhibited expression of MYDGF and culturing the ETV2-pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors; and
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils.

14. The method of claim 13, wherein the serum-free and xeno-free culture medium in (b) further comprises UM171.

15. The method of claim 13, wherein the expression of MYDGF is inhibited in the human pluripotent stem cells by gene mutation, RNA-mediated inhibition, RNA editing, DNA gene editing or base editing.

16. The method of claim 13, wherein the gene editing method comprises sequence modification using a nuclease selected from the group consisting of a meganuclease, ZFNs, TALENs, and Cas enzyme.

17. The method of claim 16, wherein the nuclease is a Cas9 enzyme.

18. The method of claim 13, wherein the retinoic acid receptor agonist is AM580.

19. The method of claim 13, further comprising step (d) selecting mature neutrophils from the population of neutrophils.

20. The method of claim 19, wherein the method comprises one or more of the following:
step (a) comprises culturing the ETV2-pluripotent stem cells for about 1 to 2 days;
step (b) comprises culturing the ETV2-HECs for about 7 to 12 days; and
steps (c) and (d) comprising culturing the myeloid progenitor cells for about 6 to 8 days.

21. The method of claim 13, wherein the pluripotent stem cells are induced pluripotent stem cells.

22. The method of claim 19, wherein the mature neutrophils can be selected by expression of CD15 and/or CD16.

23. The method of claim 19, wherein selecting comprises a cell separation, cell sorting, or enrichment method.

24. A population of neutrophils having inhibited expression of MYDGF produced by the method of claim 12.

25. (canceled)

26. A pharmaceutical composition comprising a therapeutically effective amount of the population of neutrophils of claim 24, and a pharmaceutically acceptable carrier.

27. A method of treating a microbial infection in a subject comprising administering the pharmaceutical composition of claim 26 to the subject.

28. The method of claim 27, wherein the microbial infection is a systemic infection, a bacterial infection, or a fungal infection.

29. (canceled)

30. The method of claim 26, wherein the composition is administered intravenously to the subject.

31. The method of claim 27, wherein the subject is a patient that has neutrophil deficiency, autoimmune disease, or neutrophil depletion due to radiation or chemotherapy treatment.


SEQUENCE LISTING (10 sequences)

SEQ ID NO: 1 - RNA, 19 nt, synthetic construct
  source 1..19: mol_type = other RNA
caagatactcacgtaggcc

SEQ ID NO: 2 - RNA, 20 nt, synthetic construct
  source 1..20: mol_type = other RNA
tctgtacttcacacagttca

SEQ ID NO: 3 - DNA, 22 bp, synthetic construct
  source 1..22: mol_type = other DNA
aatggcgtccatgttgagaagc

SEQ ID NO: 4 - DNA, 20 bp, synthetic construct
  source 1..20: mol_type = other DNA
gggcgctgagattgagtacg

SEQ ID NO: 5 - DNA, 24 bp, synthetic construct
  source 1..24: mol_type = other DNA
ttcctgcaagttacacaccctacc

SEQ ID NO: 6 - RNA, 1490 nt, synthetic construct
  source 1..1490: mol_type = other RNA
gcagataagcccagcttagcccagctgaccccagaccctctcccctcactccccccatgtcgcaggatcgagaccctgaggcagacagcccgttcaccaagccccccgccccgcccccatcaccccgtaaacttctcccagcctccgccctgccctcacccagcccgctgttccccaagcctcgctccaagcccacgccacccctgcagcagggcagccccagaggccagcacctatccccgaggctggggtcgaggctcggccccgcccctgcctctgcaacttgagcctggctgcgacccctgctctgacgtctcggaaaattcccccttgcccaggcccttgggggagggggtgcatggtatgaaatggggctgagacccccggctgggggcagaggaacccgccagagaacattcagaaggccttcatcgcatccatggacctgtggaactgggatgaggcatccccacaggaagtgcctccagggaacaagctggcagggcttgaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataaaaattcccggtcaaacctcaaa

SEQ ID NO: 7 - RNA, 990 nt, synthetic construct
  source 1..990: mol_type = other RNA
agtccaacatggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggacaaatatacgtgtatgttcacttacgcctctcaaggagggaccaatgagcaatggcagatgagtctggggaccagcgaagaccaccagcacttcacctgcaccatctggaggccccaggggaagtcctatctgtacttcacacagttcaaggcagaggtgcggggcgctgagattgagtacgccatggcctactctaaagccgcatttgaaagggaaagtgatgtccctctgaaaactgaggaatttgaagtgaccaaaacagcagtggctcacaggcccggggcattcaaagctgagctgtccaagctggtgattgtggccaaggcatcgcgcactgagctgtgaccagcagccctgttgcgggtggcaccttctcatctccggtgaagctgaaggggcctgtgtccctgaaagggccagcacatcactggttttctaggagggactcttaagttttctacctgggctgacgttgccttgtccggaggggcttgcagggtggctgaagccctggggcagagaacagagggtccagggccctcctggctcccaacagcttctcagttcccacttcctgctgagctcttctggactcaggatcgcagatccggggcacaaagagggtggggaacatgggggctatgctggggaaagcagccatgctccccccgacctccagccgagcatccttcatgagcctgcagaactgctttcctatgtttacccaggggacctcctttcagatgaactgggaagagatgaaatgttttttcatatttaaataaataagaacattaaaaagcaa

SEQ ID NO: 8 - protein, 342 aa, synthetic construct
MDLWNWDEASPQEVPPGNKLAGLEGAKLGFCFPDLALQGDTPTATAETCWKGTSSSLASFPQLDWGSALLHPEVPWGAEPDSQALPWSGDWTDMACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ

SEQ ID NO: 9 - protein, 81 aa, synthetic construct
PIQLWQFLLELLHDGARSSCIRWTGNSREFQLCDPKEVARLWGERKRKPGMNYEKLSRGLRYYYRRDIVRKSGGRKYTYRF

SEQ ID NO: 10 - protein, 148 aa, synthetic construct
TKTLMKKDKYTLPGGLLAPGGNSMASGVGVGAGLGAGVNQRMDSYAHMNGWSNGSYSMMQDQLGYPQHSTTAPITDVSLGDELRLDGEEVDMTPADALDDFDLEMLGDVESPSPGMTHDPVSYGALDVDDFEFEQMFTDALGIDDFGG